(19) United States
(12) Patent Application Publication
(10) Pub. No.: US 2023/0009937 A1
(43) Pub. Date: Jan. 12, 2023

(54) STAPLING END EFFECTORS FOR ENDOSCOPIC PROCEDURES

(71) Applicant: Covidien LP, Mansfield, MA (US)

(72) Inventor: Jacob C. Baril, Norwalk, CT (US)

(21) Appl. No.: 17/369,442

(22) Filed: Jul. 7, 2021

(51) Int. Cl. A61B 17/068; A61B 17/072

(52) U.S. Cl. CPC A61B 17/0686; A61B 17/072; A61B 2017/00296

(57) ABSTRACT

A surgical stapling instrument includes a releasable stapling end effector. The stapling end effector includes a body portion, a jaw assembly pivotally secured to the body portion, and an articulation mechanism extending between the body portion and the jaw assembly. The jaw assembly is articulable between a non-articulated position and at least one articulated position. The articulation mechanism includes a barrel member and first and second linkage assemblies. The barrel member defines a channel and each of the first and second linkage assemblies include a protrusion received within the channel. Rotation of the barrel member in a first direction causes the first linkage assembly to move in a proximal direction and the second linkage assembly to move in a distal direction to cause the jaw assembly to move from the non-articulated position to the at least one articulated position.

### FIELD

The disclosure relates to endoscopic surgery, and more particularly, to stapling end effectors for performing endoscopic procedures.

### BACKGROUND

Surgery often requires access to internal tissue through open or endoscopic surgical procedures. As used herein, the term “endoscopic” refers to all types of minimally invasive surgical procedures including laparoscopic, arthroscopic, natural orifice intraluminal, and natural orifice transluminal procedures. Endoscopic surgery has numerous advantages compared to traditional open surgical procedures, including reduced scarring. Endoscopic surgery is often performed within a body cavity supplied with an insufflatory fluid, e.g., carbon dioxide or saline, which provides adequate space to perform the intended surgical procedure. The insufflated body cavity is generally under pressure and is sometimes referred to as being in a state of pneumoperitoneum. Surgical access devices are often used to facilitate surgical manipulation of internal tissue while maintaining pneumoperitoneum. Trocars are often used to provide a port through which endoscopic surgical instruments are passed. Trocars generally have an instrument seal, which prevents the insufflatory fluid from escaping the body cavity while an instrument is positioned within the trocar. Alternatively, an instrument may be inserted directly through an opening, i.e., incision, in tissue into the body cavity.

The size of the instrument used during an endoscopic procedure is limited by the size of port or opening through which the endoscopic instrument is inserted. Larger ports can receive larger instruments however, larger ports require larger incisions in the patient which results in increased scar tissue. Therefore, it would be beneficial to provide an endoscopic instrument having a distal shaft with a first cross-sectional diameter that may be received either directly through tissue, or alternatively, through an instrument port, and one or more end effectors having an enlarged cross-sectional diameter that may be introduced into a cavity through an alternative means, i.e., a second larger instrument port, and may be selectively attached to and disconnected from the distal shaft within the body cavity.

### SUMMARY

A stapling end effector includes a body portion, a jaw assembly pivotally secured to the body portion, and an articulation mechanism extending between the body portion and the jaw assembly. The jaw assembly is articulable between a non-articulated position and at least one articulated position. The articulation mechanism includes a barrel member and first and second linkage assemblies. The barrel member defines a channel and each of the first and second linkage assemblies include a protrusion received within the channel. Rotation of the barrel member in a first direction causes the first linkage assembly to move in a proximal direction and the second linkage assembly to move in a distal direction to cause the jaw assembly to move from the non-articulated position to the at least one articulated position.

In certain aspects of the disclosure, the first and second linkage assemblies each include a proximal articulation link and a distal articulation link. The channel may include a first portion having a substantially “V”-shape and a second portion having a substantially inverse “V”-shape. The first portion may include first and second leg sections and the second portion may include first and second leg sections. The body portion may be configured for releasable connection to a handle assembly. The stapling end effector may further include a dynamic clamping member configured for pivoting the anvil assembly and cartridge assembly to a closed position and for ejecting surgical staples from the cartridge assembly.

The protrusion of the first linkage assembly may be disposed between the first leg sections of the first and second portions when the jaw assembly is in the non-articulated position. The protrusion of the second linkage assembly may be disposed between the second leg sections of the first and second portions when the jaw assembly is in the non-articulated position. The protrusion of the first linkage assembly may be disposed between the first and second leg sections of the first portion when the jaw assembly is in the at least one articulated position. The protrusion of the second linkage assembly may be disposed between the first and second leg sections of the second portion when the jaw assembly is in the at least one articulated position.

A surgical stapling instrument includes a handle assembly and a stapling end effector releasably secured to the handle assembly. The stapling end effector includes a body portion, a jaw assembly pivotally secured to the body portion, and an articulation mechanism extending between the body portion and the jaw assembly. The jaw assembly is articulable between a non-articulated position and at least one articulated position. The articulation mechanism includes a barrel member and first and second linkage assemblies. The barrel member defines a channel and each of the first and second linkage assemblies include a protrusion received within the channel. Rotation of the barrel member in a first direction causes the first linkage assembly to move in a proximal direction and the second linkage assembly to move in a distal direction to cause the jaw assembly to move from the non-articulated position to the at least one articulated position.

In certain aspects of the disclosure, the first and second linkage assemblies each include a proximal articulation link and a distal articulation link. The channel may include a first portion having a substantially “V”-shape and a second portion having a substantially inverse “V”-shape. The first portion may include first and second leg sections and the second portion may include first and second leg sections. The body portion may be configured for releasable connection to a handle assembly. The stapling end effector may further include a dynamic clamping member configured for pivoting the anvil assembly and cartridge assembly to a closed position and for ejecting surgical staples from the cartridge assembly.

The protrusion of the first linkage assembly may be disposed between the first leg sections of the first and second portions when the jaw assembly is in the non-articulated position. The protrusion of the second linkage assembly may be disposed between the second leg sections of the first and second portions when the jaw assembly is in the non-articulated position. The protrusion of the first linkage assembly may be disposed between the first and second leg sections of the first portion when the jaw assembly is in the at least one articulated position. The protrusion of the second linkage assembly may be disposed between the first and second leg sections of the second portion when the jaw assembly is in the at least one articulated position.

A surgical stapling instrument includes a handle assembly, an adapter assembly releasably secured to the handle assembly, and a stapling end effector releasably secured to the adapter assembly. The stapling end effector includes a body portion, a jaw assembly pivotally secured to the body portion, and an articulation mechanism extending between the body portion and the jaw assembly. The jaw assembly being articulable between a non-articulated position and at least one articulated position. The articulation mechanism includes a barrel member and first and second linkage assemblies. The barrel member defines a channel and each of the first and second linkage assemblies include a protrusion received within the channel. Rotation of the barrel member in a first direction causes the first linkage assembly to move in a proximal direction and the second linkage assembly to move in a distal direction to cause the jaw assembly to move from the non-articulated position to the at least one articulated position.

### BRIEF DESCRIPTION OF THE DRAWINGS

The accompanying drawings, which are incorporated in and constitute a part of this specification, illustrate aspects of the disclosure and, together with a general description of the disclosure given above, and the detailed description given below, explain the aspects of the disclosure, wherein:

FIG. 1 is a perspective view of a surgical stapling instrument according to aspects of the disclosure including a handle assembly, an adapter assembly, and a stapling end effector;

FIG. 2 is an enlarged view of the indicated area of detail shown in FIG. 1, with the stapling end effector separated from the adapter assembly;

FIG. 3 is a perspective side view of a stapling end effector shown in FIGS. 1 and 2, with parts separated;

FIG. 4 is a side view of a connection between a drive portion of a dynamic clamping assembly of the stapling end effector and a drive shaft of the adapter assembly;

FIG. 5 is an enlarged perspective side view of indicated area of detail shown in FIG. 3;

FIG. 6 is a perspective top view of the stapling end effector shown in FIGS. 1-3, in a non-articulated position;

FIG. 7 is an enlarged view of the indicated area of detail shown in FIG. 6;

FIG. 8 is a perspective top view of the stapling end effector shown in FIGS. 6, in an articulated position; and

FIG. 9 is an enlarged view of the indicated area of detail shown in FIG. 8.

### DETAILED DESCRIPTION

Aspects of the disclosure are described in detail with reference to the drawings, in which like reference numerals designate identical or corresponding elements in each of the several views. As used herein, the term “distal” refers to that portion of the surgical instrument, or component thereof, that is farther from the user during use of the instrument in its customary fashion, while the term “proximal” refers to that portion of the surgical instrument, or component thereof, that is closer to the user during use of the instrument in its customary fashion. As used herein, the term “clinician” refers to anyone involved in a surgical procedure, including but not limited to, surgeons, support staff, and other medical personnel. As used herein, the term “about” means that the numerical value is approximate and small variations would not significantly affect the practice of the surgical instruments. Where a numerical limitation is used, unless indicated otherwise by the context, “about” means the numerical value can vary by ±10% and remain within the scope of the disclosure.

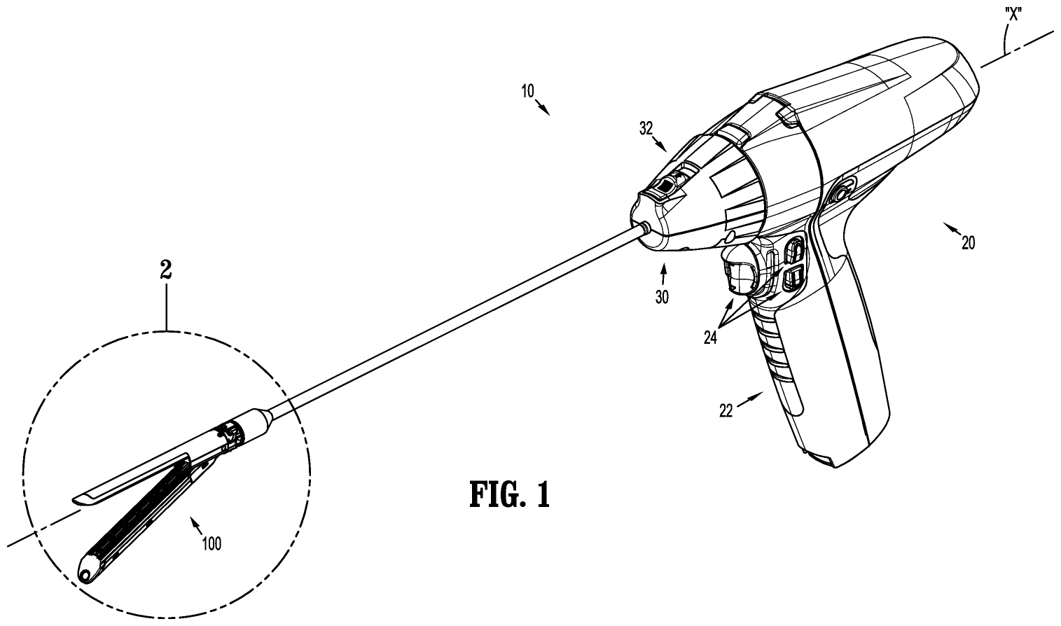

FIG. 1 illustrates an endoscopic surgical stapling instrument according to aspects of the disclosure, shown generally as surgical stapling instrument 10. The surgical stapling instrument 10 includes a handle assembly 20, and adapter assembly 30, and a stapling end effector 100. As will be described in detail below, the surgical stapling instrument 10 is configured such that the stapling end effector 100 may be attached, operated, and separated from the adapter assembly 30 during a laparoscopic procedure, and more particularly, while the stapling end effector 100 is disposed within a body cavity. Although the surgical stapling instrument 10 is shown and described for use with a stapling end effector, it is envisioned that the aspects of the disclosure may be modified for use with other types of end effectors, including those for grasping, vessel sealing, and cutting.

The handle assembly 20 of the surgical stapling instrument 10 includes a stationary grip 22. Buttons 24 on the stationary grip 22 of the handle assembly 20 allow for actuation and articulation of the stapling end effector 100. The adapter assembly 30 is releasably connected to the handle assembly 20 and includes a rotation knob assembly 32 for manual rotation of the stapling end effector 100 by a clinician about a longitudinal axis “x” of the surgical stapling instrument 10. Although the adapter assembly 30 is shown and described as being releasable from the handle assembly 20, it is envisioned that the handle assembly 20 and the adapter assembly 30 may be integrally formed.

The surgical stapling instrument 10 is illustrated as an electrically powered stapling instrument including an electrically powered handle assembly 20 that may support one or more batteries (not shown). Examples of electrically powered surgical stapling instruments can be found in U.S. Pat. Nos. 9,055,943 and 9,023,014. Alternately, it is envisioned that the surgical stapling instrument 100 could also be incorporated into a manual surgical stapling instrument such as disclosed in U.S. Pat. No. 6,238,908 or a stapling instrument that is configured for use with a robotic system, and does not include a handle assembly, such as disclosed in U.S. Pat. No. 9,962,159.

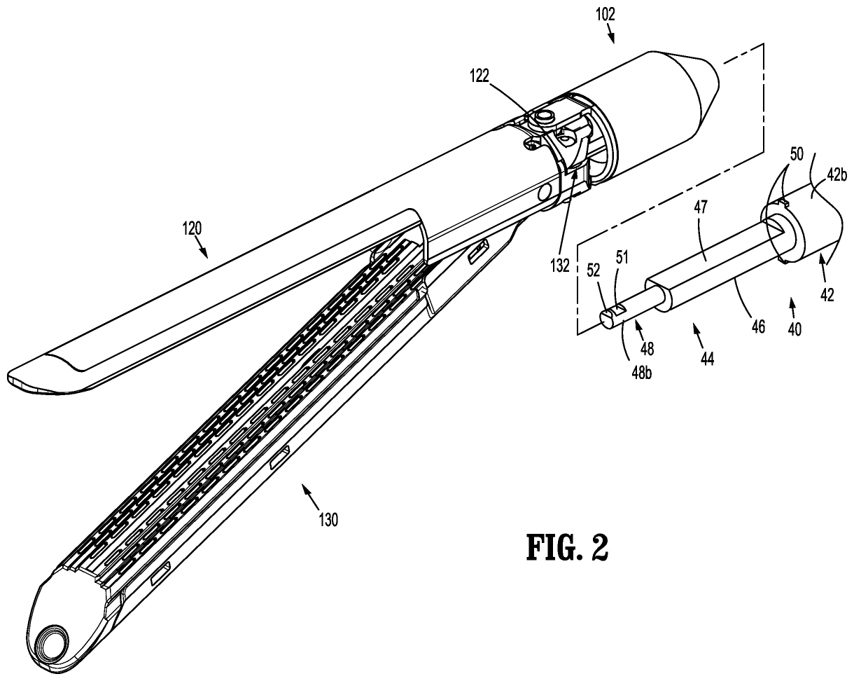

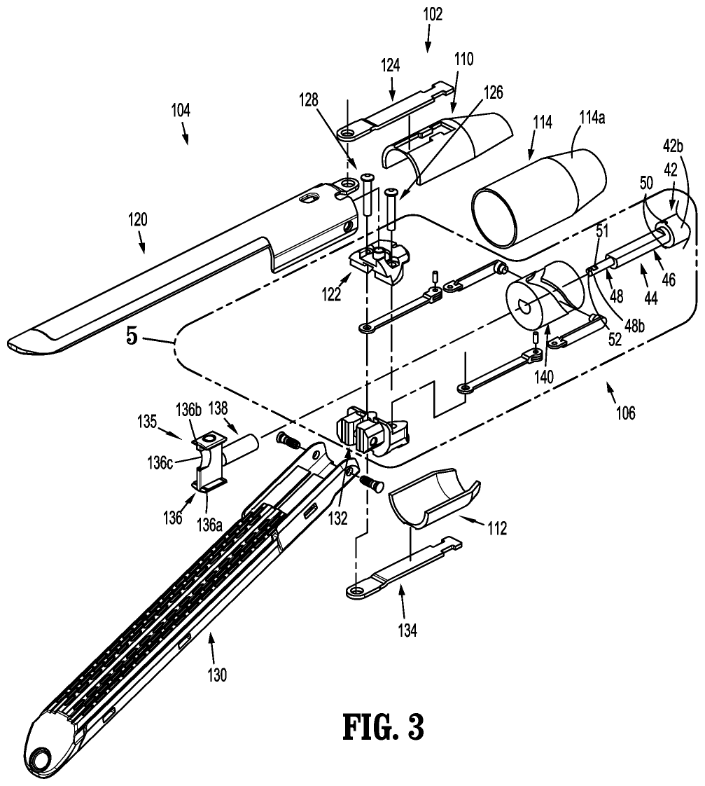

FIGS. 2 and 3 illustrate the stapling end effector 100 of the surgical stapling instrument 10 and a distal portion of an elongate body 40 of the adapter assembly 30. The elongate body 40 of the adapter assembly 30 includes a proximal outer sleeve 42 and a coaxial actuation member 44 (FIG. 3) extending through the proximal outer sleeve 42. The coaxial actuation member 44 includes an outer drive tube 46 and an inner drive shaft 48. The outer drive tube 46 and the inner drive shaft 48 move independent of each other. The outer drive tube 46 of the coaxial actuation member 44 rotates about the longitudinal axis “x” and is configured to transfer a rotational force from the handle assembly 20 to the stapling end effector 100 to effect, for example, articulation of the stapling end effector 100 relative to the elongate body 40 of the adapter assembly 30. The inner drive shaft 48 of the coaxial actuation member 44 moves along the longitudinal axis “x” and is configured to provide an axial force to the stapling end effector 100 to effect, for example, stapling of the stapling end effector 100.

A distal portion 42b (FIG. 2) of the proximal outer sleeve 42 of the elongate body 40 of the adapter assembly 30 includes tabs 50 or slots (not shown). A distal portion 46b of the outer drive tube 46 of the coaxial actuation member 44 of the elongate body 40 includes at least one flattened portion 47. A distal portion 48b of the inner drive shaft 48 of the coaxial actuation member 44 of the elongate body 40 includes a tab 52 and a notch 51 proximally spaced from the tab 52.

The stapling end effector 100 includes a base assembly 102 and a jaw assembly 104 pivotally secured relative to the base portion 102. An articulation assembly 106 extends between the base assembly 102 and the jaw assembly 104 and is configured to articulate the jaw assembly 104 relative to the base portion 102.

The base portion 102 of the stapling end effector 100 is configured for releasable connection with the elongate body 40 of the adapter assembly 30. The base assembly 102 includes upper and lower housing sections 110, 112 (FIG. 3), and a distal outer sleeve 114 receivable about the upper and lower housing sections 110, 112. A proximal portion 114a of the distal outer sleeve 114 includes slots (not shown) for receiving the tabs 50 on the outer sleeve 42 of the elongate body 40 of the adapter assembly 30. In this manner, the stapling end effector 100 is releasably engageable with the adapter assembly 30 with a bayonet-type connection. The distal outer sleeve 114 may instead include threading (not shown) for engaging corresponding threading on the proximal outer sleeve 42 or be otherwise configured for releasable engagement with the distal portion 42b of the outer sleeve 42.

The jaw assembly 104 will only be described to the extent necessary to fully disclose the aspects of the disclosure. For a detailed description of exemplary jaw assemblies, please refer to U.S. Pat. Nos. 5,752,644 and 9,016,539.

The jaw assembly 104 of the stapling end effector 100 includes an anvil assembly 120 and a cartridge assembly 130 pivotally secured relative to the anvil assembly 110. The anvil assembly 120 is secured to an upper mounting member 122 and the cartridge assembly 130 is secured to a lower mounting member 132. A first coupling member 124 connects the upper mounting member 122 with the upper housing section 112 of the body portion 102 and a second coupling member 134 connects the lower mounting member 132 with the lower housing section 114. First and second pins 126, 128 secure the upper mounting member 122 to the lower mounting member 132.

The jaw assembly 104 of the stapling end effector 100 further includes a dynamic clamping assembly 135. The dynamic clamping assembly 135 is configured to close the jaw assembly 104 and eject staples from the cartridge assembly 130. The dynamic clamping assembly 135 includes a clamping portion 136 and a drive portion 138. The clamping portion 136 and the drive portion 138 may be integrally formed or secured together in any suitable manner. The clamping portion 136 of the dynamic clamping assembly 135 includes an upper flange 136a configured to engage the anvil assembly 120 and a lower flange 136b configured to engage the cartridge assembly 130. A knife 136c is disposed between the upper and lower flanges 136a, 136b. For a detailed description of the structure and function of an exemplary dynamic clamping member, please refer to U.S. Pat. No. 10,849,621.

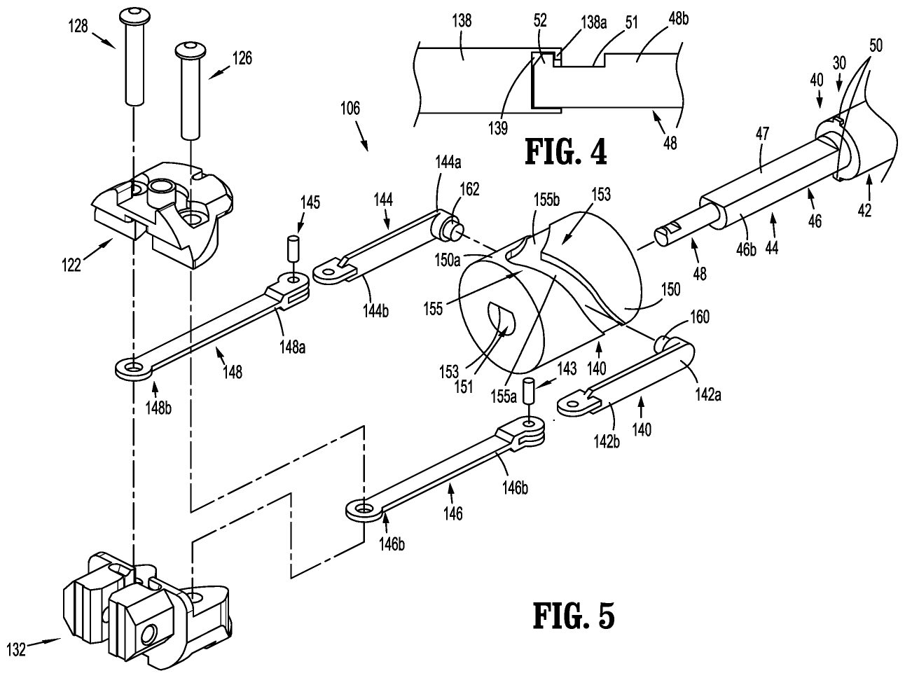

FIG. 4 illustrates a connection between the inner drive shaft 48 of the coaxial actuation member 44 of the elongate body 40 of the adapter assembly 30 and the drive portion 138 of the dynamic clamping assembly 135 of the jaw assembly 104 of the stapling end effector 100. More particularly, the drive portion 138 of the dynamic clamping assembly 135 defines a recess 139 configured to receive the distal portion 48b of the inner drive shaft 48 of the coaxial actuation member 44 of the elongate body 40 of the adapter assembly 30. A lip 138a formed on the drive portion 138 extends into the recess 139 of the drive portion 138 of the dynamic clamping assembly 136 and engages the tab 52 of the inner drive shaft 48 of the elongate body 40 of the adapter assembly 30 to secure the elongate body 40 of the adapter assembly 30 to the drive portion 138 of the dynamic clamping assembly 135 of the stapling end effector 100.

FIG. 5 illustrates the articulation mechanism 106 of the stapling end effector 100 which includes a barrel member 140, first and second proximal articulation links 142, 144, and first and second distal articulation links 146, 148. The barrel member 140 includes a substantially cylindrical body 150 defining a through bore 151 and a channel 153 formed in an outer surface 150a of the cylindrical body 150. The through bore 151 is configured to receive the distal portion 46b of the outer drive tube 46 of the coaxial actuation member 44 of the elongate body 40 of the adapter assembly 30. The through bore 151 includes a flattened portion 153 corresponding to the flattened portion 47 of the outer drive tube 46 of the coaxial actuation member 44.

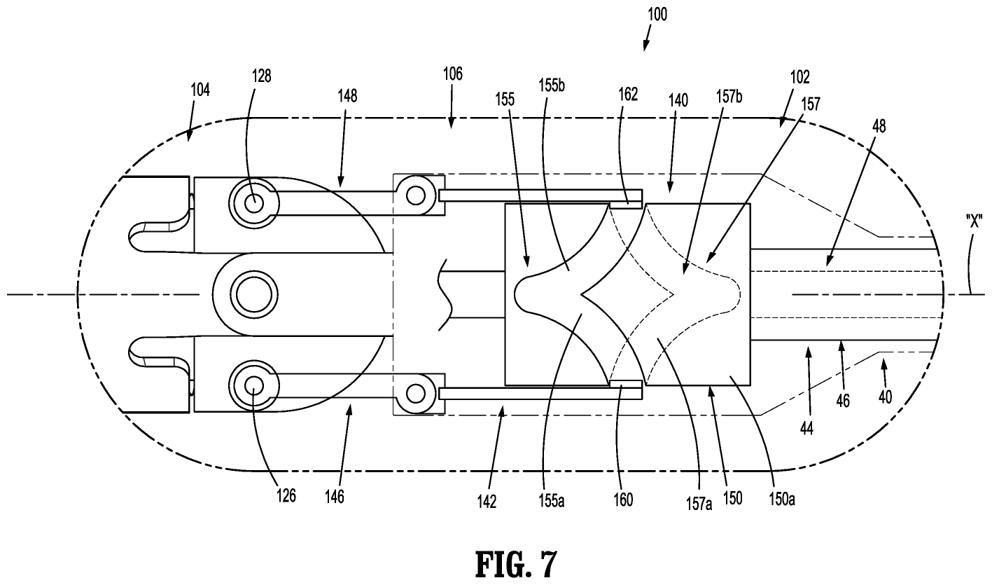

The channel 153 in the barrel member 140 of the articulation mechanism 106 includes first and second leg sections 155a, 155b forming a substantially “V” shaped first portion 155. The first and second leg sections 157a, 157b extend away from each other in a proximal direction. The channel 153 includes third and fourth leg sections 157a, 157b (FIG. 7) forming an inverted substantially “V” shaped second portion 157. The third and fourth leg sections 157a, 157b extend away from each other in a distal direction. The first leg section 155a of the first portion 155 of the channel 153 is in communication with the second leg section 157b of the second portion 155 of the channel 153 and the second leg section 155b of the first portion 155 of the channel 153 is in communication with the first leg section 157a of the second portion 155 of the channel 153.

A distal portion 142b of the first proximal articulation link 142 of the articulation mechanism 106 is pivotally secured to a proximal portion 146a of the first distal articulation link 146 with a pin 143 and a distal portion 144b of the second proximal articulation link 144 is pivotally secured to a proximal portion 148a of the second distal articulation link 148 by a pin 145. Proximal portions 142a, 144a of the first and second proximal articulation links 142, 144, respectively, include respective first and second protrusions 160, 162. The first and second protrusions 160, 162 extend inwardly and are received within the channel 153 in the barrel member 140 of the articulation mechanism 106. A distal portion 146b of the first distal articulation link 146 is pivotally secured to the upper and lower mounting members 122, 132 by the first pin 126 and a distal portion 148b of the second distal articulation link 148 is pivotally secured to the upper and lower mounting members 122, 132 by the second pin 128.

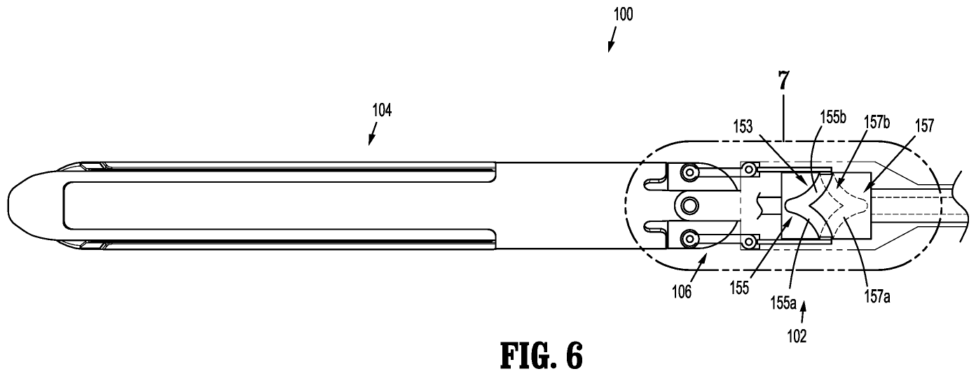

FIGS. 6 and 7 illustrate the jaw assembly 104 of the stapling end effector 100 secured to a distal portion 40b of the elongate body 40 of the adapter assembly 30 (FIG. 1) in a non-articulated position. It is envisioned that the stapling end effector 100 is received within a cavity of a patient through a suitably sized opening, e.g., access port (not shown), and the elongate body 40 of the adapter assembly 30 (FIG. 1) is received in the cavity of the patient through a second opening having a reduced diameter. In certain aspects of the disclosure, the elongate body 40 of the adapter assembly 30 is configured to be received through a five-millimeter (5 mm) access port (not shown).

When the jaw assembly 104 of the stapling end effector 100 is the non-articulated position, the barrel 140 of the articulation mechanism 106 is rotationally oriented such that the first and second protrusions 160, 162 of the respective first and second proximal articulation links 142, 144 are received within the channel 153 in the barrel member 140 such that the first protrusion 160 is positioned between the first leg section 155a of the first portion 155 of the channel 153 and the first leg section 157a of the second portion 155 and the second protrusion 162 is positioned between the second leg section 155b of the first portion 155 of the channel 153 and the second leg section 157b of the second portion 157. In this manner, the first and second protrusions 160, 162 are aligned with one another along the longitudinal axis “x”.

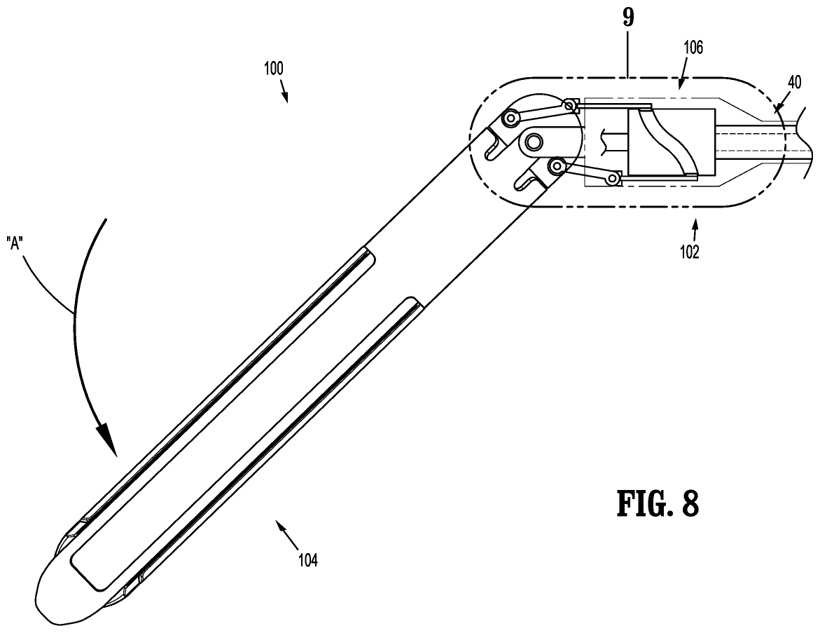

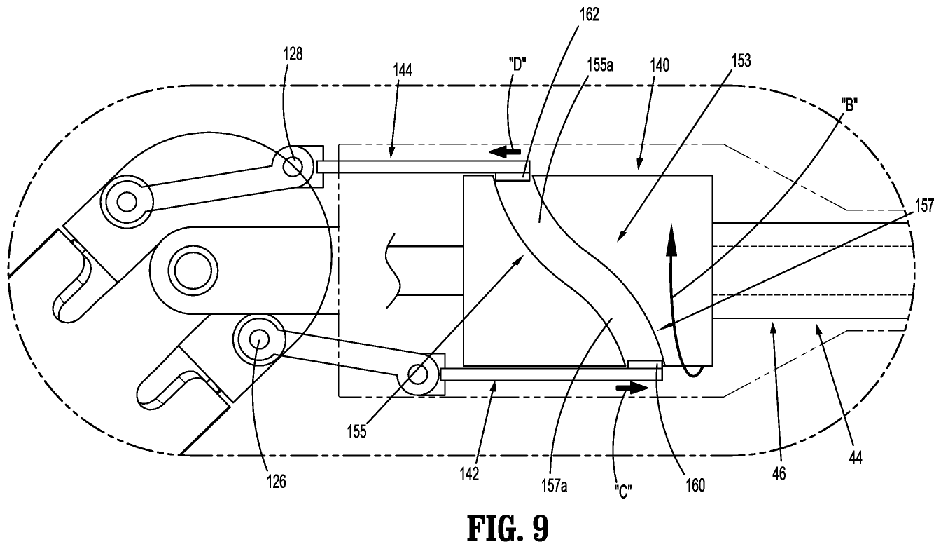

FIGS. 8 and 9 illustrate the jaw assembly 104 of the stapling end effector 100 moving to an articulated position, as indicated by arrow “A” in FIG. 8, through operation of articulation mechanism 106. Rotation of the outer drive tube 46 of the coaxial actuation member 44 causes rotation of the barrel member 140 of the articulation mechanism 106 about the longitudinal axis “x”, as indicated by arrow “B” in FIG. 9. Rotation of the barrel member 140 in the first direction causes the first proximal articulation link 142 to move proximally, as indicated by arrow “C” in FIG. 9 and causes the second proximal articulation link 144 to move distally, as indicated by arrow “D” in FIG. 9. More particularly, as the barrel member 140 rotates, the first protrusion 160 of the first proximal articulation link 142 rides along the first leg section 157a of the second portion 157 and the second protrusion 162 of the second proximal articulation link 144 rides along the second leg section 155b of the first portion 155.

When the jaw assembly 104 of the stapling end effector 100 is in a fully articulated position, the first protrusion 160 of the first proximal articulation link 142 of the articulation mechanism 106 is disposed between the first and second leg sections 155a, 155b of the first portion 155 of the channel 153 in the barrel member 140 and the second protrusion of the second proximal articulation link 146 is disposed between the first and second leg sections 157a, 157b of the second portion 157 of the channel 153 in the barrel member 140.

Conversely, when the barrel member 140 of the articulation mechanism 106 rotates in a second direction, not shown, the first protrusion 160 of the first proximal articulation link 142 rides along the second leg section 157b of the second portion 157 and the second protrusion 162 of the second proximal articulation link 144 rides along the first leg section 155a of the first portion 157 to cause the jaw assembly 104 to articulate relative to the body portion 102 in a second direction (not shown).

Actuation of the stapling end effector 100 of the surgical stapling instrument 100 is as is known in the art. Following the stapling and cutting of tissue, the stapling end effector 100 may be separated from the elongate body 40 of the adapter assembly 30 to permit removal of the elongate body 40 through a reduced diameter access port.

Persons skilled in the art will understand that the instruments and methods specifically described herein and illustrated in the accompanying drawings are non-limiting exemplary aspects of the disclosure. It is envisioned that the elements and features illustrated or described in connection with one exemplary aspect may be combined with the elements and features of another without departing from the scope of the disclosure. As well, one skilled in the art will appreciate further features and advantages of the disclosure based on the above-described aspects. Accordingly, the disclosure is not to be limited by what has been particularly shown and described, except as indicated by the appended claims.

What is claimed is:

1. A stapling end effector comprising:
a body portion;
a jaw assembly pivotally secured to the body portion and articulable between a non-articulated position and at least one articulated position; and
an articulation mechanism extending between the body portion and the jaw assembly, the articulation mechanism including a barrel member and first and second linkage assemblies, the barrel member defining a channel and each of the first and second linkage assemblies including a protrusion received within the channel, wherein rotation of the barrel member in a first direction causes the first linkage assembly to move in a proximal direction and the second linkage assembly to move in a distal direction to cause the jaw assembly to move from the non-articulated position to the at least one articulated position.

2. The stapling end effector of claim 1, wherein the first and second linkage assemblies each include a proximal articulation link and a distal articulation link.

3. The stapling end effector of claim 1, wherein the channel includes a first portion having a substantially “V”-shape and a second portion having a substantially inverse “V”-shape.

4. The stapling end effector of claim 3, wherein the first portion includes first and second leg sections and the second portion includes first and second leg sections.

5. The stapling end effector of claim 4, wherein the protrusion of the first linkage assembly is disposed between the first leg sections of the first and second portions when the jaw assembly is in the non-articulated position.

6. The stapling end effector of claim 5, wherein the protrusion of the second linkage assembly is disposed between the second leg sections of the first and second portions when the jaw assembly is in the non-articulated position.

7. The stapling end effector of claim 4, wherein the protrusion of the first linkage assembly is disposed between the first and second leg sections of the first portion when the jaw assembly is in the at least one articulated position.

8. The stapling end effector of claim 7, wherein the protrusion of the second linkage assembly is disposed between the first and second leg sections of the second portion when the jaw assembly is in the at least one articulated position.

9. The stapling end effector of claim 1, wherein the body portion is configured for releasable connection to a handle assembly.

10. The stapling end effector of claim 1, further including a dynamic clamping member configured for pivoting the anvil assembly and cartridge assembly to a closed position and for ejecting surgical staples from the cartridge assembly.

11. A surgical stapling instrument comprising:
a handle assembly;
a stapling end effector releasably secured to the handle assembly, the stapling end effector including:
a body portion;
a jaw assembly pivotally secured to the body portion and articulable between a non-articulated position and at least one articulated position; and
an articulation mechanism extending between the body portion and the jaw assembly, the articulation mechanism including a barrel member and first and second linkage assemblies, the barrel member defining a channel and each of the first and second linkage assemblies including a protrusion received within the channel, wherein rotation of the barrel member in a first direction causes the first linkage assembly to move in a proximal direction and the second linkage assembly to move in a distal direction to cause the jaw assembly to move from the non-articulated position to the at least one articulated position.

12. The surgical stapling instrument of claim 11, wherein the first and second linkage assemblies each include a proximal articulation link and a distal articulation link.

13. The surgical stapling instrument of claim 11, wherein the channel includes a first portion having a substantially “V”-shape and a second portion having a substantially inverse “V”-shape.

14. The surgical stapling instrument of claim 13, wherein the first portion includes first and second leg sections and the second portion includes first and second leg sections.

15. The surgical stapling instrument of claim 14, wherein the protrusion of the first linkage assembly is disposed between the first leg sections of the first and second portions when the jaw assembly is in the non-articulated position.

16. The surgical stapling instrument of claim 15, wherein the protrusion of the second linkage assembly is disposed between the second leg sections of the first and second portions when the jaw assembly is in the non-articulated position.

17. The surgical stapling instrument of claim 14, wherein the protrusion of the first linkage assembly is disposed between the first and second leg sections of the first portion when the jaw assembly is in the at least one articulated position.

18. The surgical stapling instrument of claim 17, wherein the protrusion of the second linkage assembly is disposed between the first and second leg sections of the second portion when the jaw assembly is in the at least one articulated position.

19. The surgical stapling instrument of claim 11, wherein the body portion is configured for releasable connection to a handle assembly.

20. A surgical stapling instrument comprising:
a handle assembly;
an adapter assembly releasably secured to the handle assembly; and
a stapling end effector releasably secured to the adapter assembly, the stapling end effector including:
a body portion;
a jaw assembly pivotally secured to the body portion and articulable between a non-articulated position and at least one articulated position; and
an articulation mechanism extending between the body portion and the jaw assembly, the articulation mechanism including a barrel member and first and second linkage assemblies, the barrel member defining a channel and each of the first and second linkage assemblies including a protrusion received within the channel, wherein rotation of the barrel member in a first direction causes the first linkage assembly to move in a proximal direction and the second linkage assembly to move in a distal direction to cause the jaw assembly to move from the non-articulated position to the at least one articulated position.
